(19) United States
(12) Patent Application Publication
(10) Pub. No.: US 2006/0065528 A1
(43) Pub. Date: Mar. 30, 2006

(54) Nanostructured devices for separation and analysis

(76) Inventors: Gabriel Lopez, Albuquerque, NM (US); Steven R.J. Brueck, Albuquerque, NM (US); Linnea K. Ista, Albuquerque, NM (US); Michael O'Brien, Albuquerque, NM (US)

Correspondence Address: SCHWEGMAN, LUNDBERG, WOESSNER & KLUTH, 1600 TCF TOWER, 121 SOUTH EIGHT STREET, MINNEAPOLIS, MN, 55402, US

(21) Appl. No.: 11/050,424

(22) Filed: Feb. 3, 2005

Related U.S. Application Data
(60) Provisional application No. 60/541,438, filed on Feb. 3, 2004.

(51) Int. Cl. B01L 3/00; C07K 1/26

(52) U.S. Cl. 204/450; 204/600; 422/99

(57) ABSTRACT

Methods for forming an apparatus containing a nanofluidic device with a pattern having nanoscopic features includes producing a regular interference pattern in a substrate using two coherent light beams. In an embodiment, an apparatus includes a nanofluidic device having nanoscopic features in at least two dimensions. In an embodiment, a nanofludic device having nanoscopic features is formed using an ultraviolet source to generate a regular interference pattern.

### RELATED APPLICATIONS

This application claims priority under 35 U.S.C. 119(e) from U.S. Provisional Patent Application Ser. No. 60/541,438 filed 3 Feb. 2004, which application is incorporated herein by reference.

This application is related to the following, commonly assigned application, U.S. patent application Ser. No. 10/073,935, entitled “Nanostructured Devices For Separation And Analysis,” now U.S. Pat. No. 6,685,841, which claimed priority to U.S. Provisional Patent Application Ser. No. 60/268,365, entitled “Nanostructured Devices for Separation and Analysis,” filed on Feb. 14, 2001, the entire contents and disclosure of both are incorporated herein by reference. This application is related to the following, commonly assigned application, U.S. patent application Ser. No. 10/338,654, entitled “Nanostructured Separation and Analysis Devices For Biological Membranes,” filed Jan. 9, 2003.

### GOVERNMENT INTEREST STATEMENT

This invention is made with government support under grant number DAAD19-99-1-0196 awarded by the United States Army Research Office. The government may have certain rights in this invention.

### FIELD OF THE INVENTION

The present invention relates generally to nanostructures, and more particularly to the fabrication and use of nanostructures for separation and analysis of molecules.

### BACKGROUND

Polyacrylamide gel electrophoresis (PAGE) remains the standard for protein separation and identification in biotechnology. Nevertheless, the set of separation strategies that rely on this technique are hampered by: (1) inconvenience of preparation of the variety of gels needed for the separations, (2) inherent inconsistencies in production conditions; and therefore, irreproducibility between different batches of gels, (3) limited resolution and dynamic range of biomolecular separations, (4) susceptibility of the polymer to degradation under high electric fields, (5) lack of reusability, and (6) difficulty in incorporation of these techniques into strategies for development of multidimensional (multi-technique) integrated separation systems.

Gradient PAGE techniques are recognized to have the potential to have excellent resolution and dynamic range, but their utility is greatly hampered by the need for cumbersome gel preparation protocols and lack of reproducibility.

The demand for precise separation of molecules using small sample volumes is increasing. Separation of molecules across matrices or membranes has been known for long in the art. Separations are generally achieved by employing barriers that allow cutoffs at a precise molecular weight or by size-exclusion. The art describes structures where molecular transport and filtration take place perpendicular to the surface of the separating material. The currently available systems, however, suffer from a number of drawbacks. For example, biomolecules may not be amenable to separation by many of the available systems. For example, reaction steps may denature or inactivate the molecules themselves. The matrices formed are generally composed of non-uniform structures. Even where a gradation in size of structures is required, they may be random or at best have to be serially and sequentially arrayed through a cumbersome process of lithography. Fabrication of such separation devices also poses problems in terms of batch-to-batch variations and consequently poor reproducibility of results therefrom. Lack of efficiency of separation or loss of sample volume is also encountered.

Nano-filtration of molecules using “Brownian ratchets” in which assymetric diffusion leads to separation of molecules based on their size (van Oudenaarden et al. Science, 285: 1046-1052, 1999) has been tried with some success. Chou et al., Proc. Natl. Acad. Sci. 96, 13762-13765, 1999, attempted separation of DNA molecules using microsystems formed by conventional photolithography. However, the developments have not gained ground with users primarily because of the difficulty of preparation of the nanofluidic systems and the associated high-cost of fabrication. Other separation matrices such as gradient polyacrylamide gels, where one-dimension filtration was achieved by manipulating pore-size through control of cross-linker, monomer and solvent concentrations, has shown limited success. Even though the separation is effective, the preparation process is tedious and the results obtained are not reproducible. “Artificial gels” incorporating regular arrays of nanoscale pillars created through electron beam and/or imprint lithography have been described, for example, in U.S. Pat. No. 6,110,339 to Brueck et al. and by Turner et al. (J. Vac. Sci. Technol. B., 16 3835-3840, 1998). All these nanolithographically-defined structures utilize regular arrays of uniform-sized nanostructures throughout the separation matrix. Thus, the systems suffer from resolution and flexibility limitations. It is also difficult to integrate such a system with other more complex separation devices. Thus, the need for an efficient, highly-resolving, flexible, cost-efficient and reproducible molecular separation matrix is largely unmet.

### SUMMARY

The above mentioned problems are addressed by the present invention and will be understood by reading and studying the following specification. In an embodiment, a method includes producing a regular interference pattern in a substrate using two coherent light beams to form a nanofluidic device having a pattern with nanoscopic features in at least two dimensions. In an embodiment, an apparatus includes a nanofluidic device in a substrate, where the nanofluidic device has a structure that is nanoscopic in two dimensions.

These and other embodiments, aspects, advantages, and features of the present invention will be set forth in part in the description which follows, and in part will become apparent to those skilled in the art by reference to the following description of the invention and referenced drawings or by practice of the invention. The aspects, advantages, and features of the invention are realized and attained by means of the instrumentalities, procedures, and combinations particularly pointed out in the appended claims.

### BRIEF DESCRIPTION OF THE DRAWINGS

Embodiments are described in conjunction with the accompanying drawings.

FIG. 1 is a micrograph showing an 150-nm period photoresist grating written with 213 nm light.

FIG. 2 is a micrograph showing 30-nm photoresist lines.

FIG. 3 is a micrograph showing a 108-nm pitch photoresist grating, written using 213 nm light, and immersion in DI water.

FIG. 4 is a micrograph showing a photoresist line interpolated between two lines etched 360 nm apart into a nitride film demonstrating spatial period division to extent the spatial frequency coverage of optical lithography.

FIGS. 5A and 5B are micrographs showing transfer of interferometric lithography patterns into deep structures in Si using KOH anisotropic etching, with FIG. 5A showing the original period of 360 nm with about 1 micrometer deep etched grooves and FIG. 5B showing the 180 nm period, frequency-doubled structure corresponding to the lithographic result of FIG. 4.

FIG. 6 illustrates in schematic form a nanostructured gradient (chirped) separation matrix.

FIGS. 7A and 7B show perspective and top schematic views, respectively, of an embodiment of a nanostructured matrix.

FIGS. 8A, 8B and 8C show high aspect ratio nanostructures fabricated by interferometric lithography and pattern transfer with FIG. 8A showing dense 150 nm photoresist lines, FIG. 8B showing an isolated 50 nm photoresist line, and FIG. 8C showing 50 nm wide walls etched in Si.

FIG. 9 is a schematic of a purification chip containing several biomolecular sieves with different aperture sizes.

FIGS. 10A and 10B are schematics depicting embodiments of monolithic multi-technique separation systems with FIG. 10A showing a 2-technique, (2-dimensional) separation in a single level separation system and FIG. 10B showing an exploded view of a 2-technique separation in a two-level separation system.

FIG. 11 is a schematic of a simple electrophoretic cell that incorporates a nanofluidic separation matrix patterned using IL.

FIG. 12 shows an embodiment of a right angle reflector assembly for exposure.

FIGS. 13A-13G illustrate an embodiment of a method including mask fabrication.

FIGS. 14A-14C show FEM images of etched samples for an embodiment.

FIGS. 15A-15D show confocal images of fluid motion in an embodiment of a large-area grating design.

FIGS. 16A-16D show confocal microscopy of electrophoresis in an embodiment of an integrated chip.

### DETAILED DESCRIPTION

In the following detailed description of the invention, reference is made to the accompanying drawings that form a part hereof, and in which is shown, by way of illustration, specific embodiments in which the invention may be practiced. These embodiments are described in sufficient detail to enable those skilled in the art to practice the invention. Other embodiments may be utilized and structural, logical, and electrical changes may be made without departing from the scope of the present invention.

### DEFINITIONS

For the purposes of the present invention, the term “nanostructure” refers to a protrusion or void having a diameter in at least one direction of 1 to 500 nm.

For the purposes of the present invention, the term “diameter” refers to the distance across a nanostructure through the middle and perpendicular to the axis of the nanostructure, parallel to the plane of the substrate (upon which the nanostructure is located).

For the purposes of the present invention, the term “axis” refers to a line running along the middle of a nanostructure in the direction the nanostructure's longest dimension parallel to the surface of the substrate on which the nanostructure is located.

For the purposes of the present invention, the term “protrusion” refers to a structure that protrudes from the surface of a substrate or that protrudes from a portion of a substrate that has been etched. The protrusions of the present invention may be any convenient size or shape. The cross-section of a protrusion may be circular, square, rectangular, oval, elliptical, etc.

For the purposes of the present invention, the term “channel” refers to a gap between any two protrusions. The channels of the present invention may be any convenient size or shape.

For the purposes of the present invention, the term “array” refers to an arrangement of nanostructures.

For the purposes of the present invention, the term “gradient” refers to an array where channels, protrusions or other features at one end of the array are larger than those at an opposite end of the array.

For the purposes of the present invention, the term “continuous gradient” refers to a gradient where successive rows of channels, protrusions or other features decrease in size substantially continuously from one end of the gradient to the other end of the gradient.

For the purposes of the present invention, the term “non-continuous gradient” refers to a gradient that includes regions of the gradient having successive rows of channels, protrusions or other features that are substantially the same size.

For the purposes of the present invention, the term “matrix” refers to a substrate having an array of nanostructures present on or in at least a portion of the substrate. A matrix of the present invention preferably has at least one gradient on or in the substrate formed by the nanostructures. Examples of a matrix of the present invention include one or more arrays located on a chip, such as a semiconductor chip, biochip, etc. Methods for making biochips which may be readily adapted for use in making biochips of the present invention are described in U.S. Pat. No. 6,174,683, the entire contents and disclosure of which is hereby incorporated by reference.

For the purposes of the present invention, the term “interferometric lithography” (IL) refers to a process of lithography that involves interference patterns of two (or more) mutually coherent light waves. The angles between the light propagation vectors of the waves are sufficiently large to produce an interference pattern that has a high spatial frequency. The resulting interference pattern may have nanoscale dimensions. Examples of interferometric lithography techniques that may be used in the present invention are described in Chen X L, Brueck S R J, “Imaging interferometric lithography: approaching the limits of optics” in Optics Letters, 24, pp. 124-126 (1999), in “Imaging interferometric lithography: A wavelength division multiplex approach to extending optical lithography, Chen X L, Brueck S R J, Journal of Vacuum Science and Technology B, vol. 16, pp. 3392-3397 (1998), in U.S. Pat. No. 5,759,744 to Brueck et al., in U.S. Pat. No. 6,233,044 to Brueck et al., and U.S. Pat. No. 6,042,998 to Brueck et al., the entire contents and disclosures of which are hereby incorporated by reference.

For the purposes of the present invention, the term “biomolecules” refers to biologically derived macromolecules such as peptides, small polypeptidess, long polypeptides, proteins, antigens, antibodies, tagged proteins, oligonucleotides, nucleotides, polynucleotides, aptamers, DNA, RNA, carbohydrates, etc. and complexes thereof.

For the purposes of the present invention, the term “size exclusion separation process” refers to separating particles, such as biomolecules, by size based on the ability of smaller particles to pass through smaller openings or channels than larger particles.

For the purposes of the present invention, the term “gel electrophoretic mobility separation process” refers to any conventional electrophoresis separation technique such as two-dimensional polyacrylamide gel electrophoresis. Polyacrylamide gel electrophoresis (PAGE) is used to separate biomolecules, usually proteins or DNA fragments, by the ratio of each biomolecule's mass to charge. Proteins may be separated in either their native state, or denatured by the addition of a detergent such as SDS (Sodium Dodecyl Sulfate). Further resolution may be obtained in some cases by making a gel with a gradient either in the concentration of the acrylamide or in the degree of crosslinking within the gel matrix. The array of the present invention may be used to doing equivalent molecular weight separations, with either electrical currents or flow as the drive force.

For the purposes of the present invention, the term “isoelectric focusing separation process” refers to the separation of charged biomolecules, such as proteins and peptides, by the each biomolecule's isoelectric point. A pH gradient is generally generated using a mixture of ampholytes within the separation matrix, usually polycrylamide. The biomolecules in the mixture then migrate to the region where the pH is equal to a particular biomolecule's isoelectric point, at which time the charged biomolecules become electrically neutral. This technique, combined with subsequent separation by SDS-PAGE, is used in traditional two-dimensional gel electrophoresis. Similar pH gradients may be generated using an array of the present invention including a two-dimensional gradient, using traditional isolectric focusing with soluble ampholytes or by using chemical patterning techniques, or immobilization of ampholytes after electrical focusing. Examples of capillary-based isoelectric focusing separation processes suitable for use with the present invention are described in Thorman, Tsai, Michaud, Mosher and Bier “Capillary Isoelectric-Focusing: Effects of Capillary, Geometry, Voltage Gradient and Addition of Linear Polymer” J. Chromatography, 398:75-86 (1987), the entire contents and disclosure of which are hereby incorporated by reference.

For the purposes of the present invention, the term “asymmetric diffusion separation process” refers to a separation process in which steric constraints drive diffusion preferentially in one direction. Examples of asymmetric diffusion separation processes suitable for use with the present invention are described in Van Oudenaarden et al., Science, 285: 1046-1052 (1999), the entire contents and disclosure of which are hereby incorporated by reference.

For the purposes of the present invention, the term “entropic trapping separation process” refers to separations using nanostructured devices of alternating thin and thick regions, with the thin regions being smaller than the radius of gyration of the biomolecule being separated. Under an electrical field, the molecules repeatedly change conformation, costing entropic free energy, thus limiting mobility. An example of an entropic trapping separation process suitable for use with the present invention is described in Han J, Craighead H D, “Separation of long DNA molecules in a microfabricated entropic trap array” Science, 288: 1026-1029 (2000), the entire contents and disclosure of which is hereby incorporated by reference.

For the purposes of the present invention, the term “hydrophobic interaction chromatography separation process” refers to a technique whereby molecules are partitioned between a hydrophobic matrix and a hydrophilic solvent. The degree of hydrophobicity of the target molecule determines the target molecule's retention time. The array of the present invention may be modified to incorporate a gradient of hydrophobicities or to create a milieu in which the hydrophobicity may be rapidly and reversibly changed, thus providing a driving force for molecular movement.

For the purposes of the present invention, the term “affinity chromatography separation process” refers to a chromatography process that takes advantage of specific chemical interactions between a target molecule and a chromatographic matrix. One of the most widely used forms of affinity chromatography employs immunoaffinity in which an antibody or series of antibodies are immobilized on a support. Other affinity agents include enzymes that interact with specific targets or receptors. Another example of affinity chromatography is a molecular recognition separation process such as the separation of long DNA molecules in a micro fabricated entropic trap array. An array of the present invention may be used for both the generation of affinity matrices and for the subsequent use of affinity matrices.

For the purposes of the present invention, the term “enantiomeric resolution separation process refers to a process to separate organic particles, such as biomolecules by chirality. Enantiomeric resolution is especially important in carbohydrate separations where differences between different glycosides are exclusively enantiomeric. Indeed, common chiral selectors are cyclodextrins used in capillary electrophoresis. Macrocyclic antibiotics and crown ethers are commonly used selectors. Selectors may be used either be used either globally or in zones of the array of the present invention to confer yet another means of separation.

For the purposes of the present invention, the term “capillary electrophoresis separation process” refers to a separation process in which separation takes place in a liquid rather in a gel matrix. Capillary electrophoresis allows for separations to be done on smaller quantities of material and with improved resolution in comparison to convention gel electrophoresis processes. The channels in an array of the present invention may be arranged to generate a capillary type arrangement in a second direction following separations based on chemical properties (e.g., IEF, affinity, hydrophobic interaction chromatography or enantiomeric separation) or capillaries may be applied as a third dimension.

For the purposes of the present invention, the phrase “comprises Si” refers to silicon and any silicon complex, compound, etc. that includes silicon, such as SiO₂, glass, etc.

Various embodiments provide:

a highly-efficient and facile nanostructured matrix for separation and analysis of molecules;

a matrix that enables gradient or non-uniform transport of molecules across a plane parallel to the surface of the matrix;

a means to enable integration of multi-dimensional multi-technique molecular separation systems into a single platform;

a means for customized fabrication of a nanostructured separation matrix including an array having a gradient property;

a nanostructured matrix that may be easily cast to cater to different ranges of molecular separations, in terms of resolution and dynamics;

a means to enable uniform consistency in the composition of the nanostructures forming the separation matrix;

a means to enable separation and/or identification of a molecular species;

a means to enable calibration-free use of the separation/analysis process;

a means to enable multiple use of a single separation matrix;

a means to enable parallel production of separation matrices at relatively low cost;

a means to provide enhanced reproducibility and resolution in the separation of molecules;

a matrix comprising an array of nanostructures arranged so that the array has a gradient property;

a method for forming an array having a gradient property including: (a) providing a substrate; and (b) forming nanostructures on the substrate to form an array having a gradient property; and/or

a separation method including: (a) providing a matrix comprising an array having a gradient property, the array comprising nanostructures; and (b) conducting at least one biomolecule separation process to separate biomolecules in a composition containing a plurality of biomolecules using the matrix.

Various embodiments provide, in part, for robust, inexpensive and reproducible methods for forming separation matrices for gradient separations based on, for example, electrophoresis and size exclusion that will have all the positive traits of gradient PAGE. These matrices may be adapted for a host of variant separation strategies, including electrophoresis, detergent solubilization, native electrophoresis, isoelectric focusing, 2D-electrophoresis, hydrophobic interaction, and affinity chromatography. The methods of fabrication discussed herein may also be adapted by existing microfabrication and integration facilities.

Various embodiments provide for separation of molecular species across a nanostructured matrix, a method of fabricating nanostructures comprising the matrix and the use of such a matrix for separation and/or analysis of molecules by defining the physical size and/or chemical features of the nanostructures as a means of screening. Various embodiments may be used to separate biological materials, such as proteins, carbohydrates, and nucleic acids as well as nonbiological materials, such as synthetic polymers. These nanostructures may be made out of a variety of materials, including silicon, thus providing systems that may be easily chemically modified for additional flexibility. The use of lithography to generate nanostructured separation matrices has advantages over other techniques (such as traditional acrylamide gel polymerization) since it (1) creates highly ordered structures, (2) gives the possibility of creating macroscopic arrays of continually varying size or chemistry across one dimension, (3) is highly reproducible, and (4) may be easily implemented in the creation of complex, integrated separation systems that are disposable or reusable. Furthermore, the use of lithographically defined separation matrices lends itself to the facile implementation of these matrices into multi-level, 3-dimensional separation devices in which different screening mechanisms allow enhanced separations. Various embodiments aim to eliminate some of the current limitations by the fabrication of highly uniform and reproducible nanostructured separation systems prepared by nano- and microlithography.

Nanolithographically-Defined Gradients:

Using an advanced lithographic technique such as interferometric lithography (IL) capable of producing nanostructures, patterns of nanostructures may be rapidly created over wide, macroscopic areas at low cost (compared to other techniques such as electron beam lithography). In addition, it may be used to easily generate arrays of nanostructures (protrusions or channels) whose dimensions vary semi-continuously in the plane of surface of the material being patterned. IL has advantages over other methods that might be used to construct nanopatterned fluidic structures (e.g., electron beam lithography, X-ray lithography, or local probe lithography) due to the low cost of implementation and the parallel nature of the lithographic technique. Combining IL with conventional lithography allows for the formation of device structures in individual areas and adding aperiodic features such as electronic and fluidic connections. Imaging interferometric lithography extends optics to fundamental, deep-subwavelength scales.

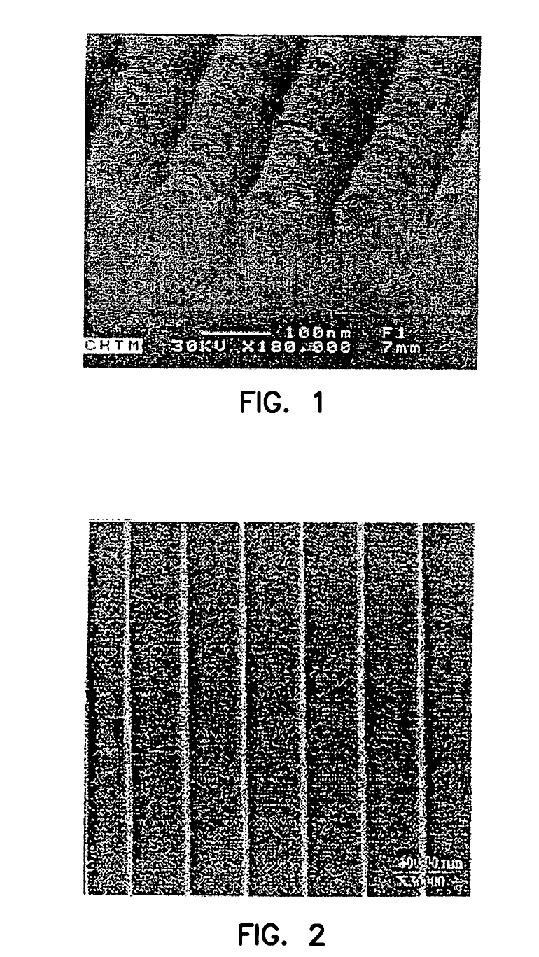

It is worthwhile at this point to consider the fundamental limits of optical lithography. For the interference of two plane waves in air, the period is given by λ/(2sin θ) where λ is the optical wavelength and θ is the angle of incidence. For a 213-nm laser source (fifth harmonic of YAG) this gives a period of ˜150 nm (for θ=80°). FIG. 1 shows an example of a large-area, 150 nm period, photoresist grating. It is important to realize that this limit is on the period, not on the feature dimensions. Nonlinearities in the exposure/develop processes and in subsequent processing may reduce the feature to dimensions well below λ/4. An example in FIG. 2 shows 30-nm developed resist lines on a 360-nm pitch written at a wavelength of 364 nm. The ultimate limit in linewidth is set by material properties and by uniformity of the processing; linewidths as small as 10 nm are routinely achieved. The use of immersion techniques, may further reduce the period by a factor of the refractive index, approximately a factor of 1.5, to a period of ˜75 nm. Initial results reproduced the 150 nm pitch of FIG. 1 at a lower angle of incidence.

Water and higher-index liquids, including liquid Ar (n˜1.6) may be used to further extend these results into the sub-100-nm period regime that will be important for biological separations. FIG. 3 shows an initial example of immersion interferometric lithography where the grating period has been reduced to 108 nm with exposure by 213 nm light using immersion in deionized water.

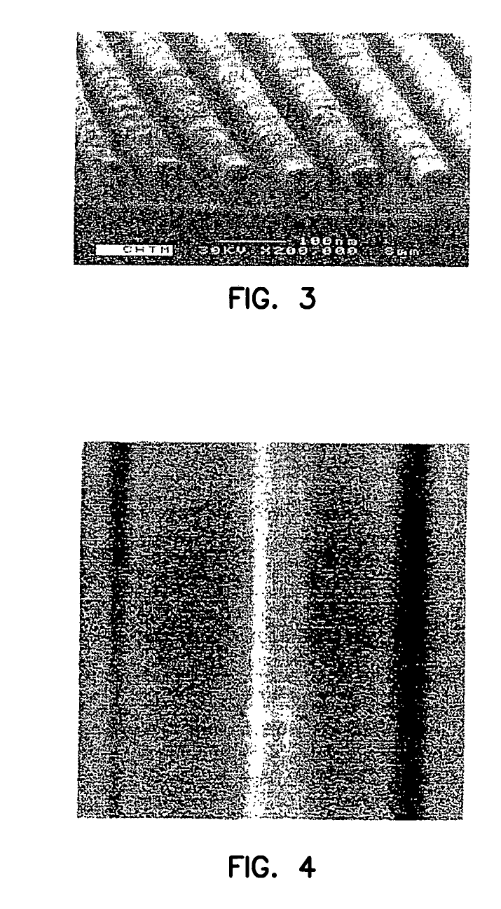

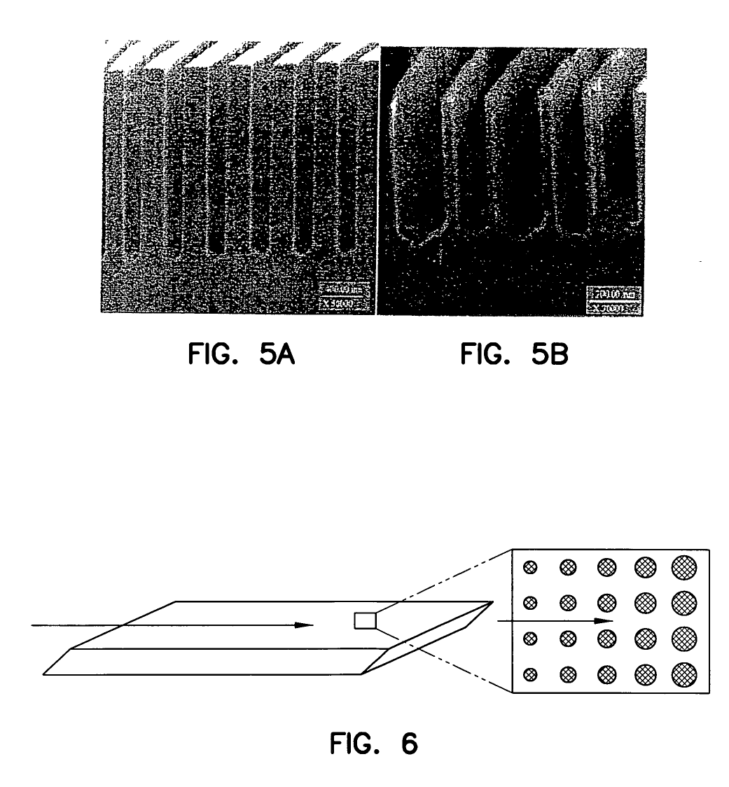

Nonlinear processes may be used to further reduce the period. FIG. 4 shows an example of a photoresist line interpolated between two parallel lines that have already been transferred into a nitride layer. FIG. 5B shows the result of transferring both of these patterns into Si using a KOH etch process. The final period is ˜half of the initial IL period. Extending the calculation above with this spatial period division gives a period of ˜37 nm and a dense linewidth of ˜17 nm (λ/12).

Importantly, all of these results are macroscopic in scale, e.g., covering areas of ˜1 cm² or larger. A strength of optics is the parallel nature of the exposure, 20 which may be cm's or larger in extent. For a square lattice with a 100-nm pitch and a 1 cm field, there are 10¹⁰ features, well beyond the realistic capabilities of serial techniques such as e-beam and scanning probes. In particular embodiments of the present invention, IL may be extended deep into the nanometer regime (either to feature sizes of ˜10 nm or nearest-neighbor distances (aperture sizes) of <10 nm, but not both simultaneously).

A continuously varying channel spacing between nanostructures is desired for many of the bio-separation applications such as various nanofluidic configurations discussed herein.

One approach to a graded structure is to macroscopically vary the intensity across the plane of exposure while keeping the other interference conditions, such as the angles between the light propagation vectors and the polarization, unchanged. One such variation of intensity would be a smooth gradient in intensity of one of the two interfering light waves. This results in interference fringes with uniform spacing but different intensities. The difference in intensity of the fringes leads to differences in exposure of the photoresist used. Because the fringe spacing is not changed, the pitch is uniform. The interference pattern would have even better contrast if both light waves had the same gradient in intensities.

When a positive photoresist is used, the areas corresponding to fringes with stronger intensities leave wider cavities in the photoresist after exposure and developing. The areas corresponding to fringes with weaker intensities leave narrower cavities in the photoresist. When the substrate is etched, these differing widths translate into features in the substrate that have differing widths. The features have the same pitch, however, because the fringe spacing is not altered. This leads to a constant pitch, but a varying line:space ratio. This procedure provides a continuously decreasing channel width that may be accurately controlled over very long distances. Such gradient separation matrices exhibit the favorable traits of gradient gels (high resolution in separation), without the difficulty and irreproducibility associated with their preparation

Similarly, this technique when used with negative photoresist leaves wider features in the areas corresponding to fringes with weaker intensity and narrower features in the area corresponding to fringes with stronger intensity.

An alternative approach may produce features with a gradient in width and pitch. This may be easily achieved with IL by using a cylindrical lens in one of the beams, while keeping the other beam as a plane wave. In this case the plane of exposure becomes a chord for a number of circular wavefronts. Because the wavefronts have different radii of curvature (spacing of an optical wavelength), the spacing between the interference fringes, as well as the width of the interference fringes, vary along the length of the plane containing the interference fringes on the surface of the photoresist coating the substrate. Similarly, curved surfaces (sections of Newton's rings) may be formed by interfering a plane wave and a spherical wave or two spherical waves of differing radii of curvature.

Other types of separation systems may involve discontinuous gradients. One such system may have differing aperture sizes that may be produced by separate exposures with different intensities, at different pitches through shadow masks, or by using multiple exposure techniques to eliminate rows and/or columns of pillars in certain areas of a previously exposed uniform nano-structured surface.

Variations in size may also be produced chemically. For example, increasing the oxidation of silicon in certain areas of a chip will result in a swelling of the features, reducing the width of some channels while conserving the pitch of the features. Similarly, macroscopic areas may be selectively functionalized with monolayers, reducing the width of channels contained in that area.

One may also electrochemically produce silicon carbide on a silicon substrate. Silicon carbide is suitable for sublimation growth, allowing one to control the width of the modified channels in a certain area. Of course, silicon carbide is only one example of surface modifications that can be performed.

One may also selectively heat a substrate, bringing it close to its annealing temperature. At this time the substrate may be placed under a highly controlled stress. The subsequent strain alters the size of channels. A gradient in temperature across the substrate results in a gradient of strain, and therefore a gradient in channel widths. This technique would only be suitable for substrates without a crystalline structure (such as glass or amorphous silicon, for example).

The very high aspect ratios of FIGS. 5A and 5B were achieved using highly anisotropic wet chemical etching of crystalline Si in KOH, which exhibits a >400:1 etch-rate selectivity for etching the <100> plane relative to the <111> plane of Si. Thus, the vertical sidewalls are nearly perfect <111> Si facets. These structures may be further modified by oxidation. This provides insulation between the Si and the surrounding material (allowing electrophoretic fluidic manipulation) and varies the surface interactions between the nanostructure and the surrounding materials for fluidic applications. Very high aspect ratio, crystal-structure-independent etching processes have been developed to address the need for 3D structures in MEMs technology. These involve pulsed gas processes in which an isotropic etch process is alternated with a surface passivation step to reduce the sidewall etch rate and only etch feature bottoms exposed by ion bombardment. To date, these processes have largely been investigated on micrometer scales. Various embodiments provide processes at the nanostructured regime. This greatly broadens the available classes of materials for which deep, high aspect ratio structures suitable for nanofluidic applications may be fabricated.

Nanostructures that exhibit a gradient in their capacity to transport biomolecular species (through size exclusion or otherwise) may be created by the IL processes discussed herein. Such gradients make separation matrices feasible for highly efficient separation of molecular species. Molecular species may be driven in the direction of the gradient, and thus separated based on their tendency to traverse the gradient, by a variety of driving forces, including, but not limited to, electrophoresis, externally-applied pressure, capillarity, diffusion, and osmosis.

IL represents a convenient method for generating nanostructured separation matrices that contain physical gradients that allow selective transport of chemical species and, thus, may be used to achieve a separation of different chemicals. When compared to other nanolithographic methods of pattern generation (e.g., electron beam lithography, scanning probe lithography), it is more convenient, efficient and inexpensive because it may be used to generate the entire pattern in one, parallel step and is not a serial “writing” technique. Other parallel techniques (e.g., imprint lithography) rely on a primary patterning technique to generate a master that may then be used to produce replicas of nanostructured features in a parallel fashion. While IL is a preferred method to generate nanostructured gradients for molecular separation, a variety of methods could be employed to generate the nanostructured matrix gradient “artificial gels” of the present invention. Gradients in the chemistry of the separation matrix may be prepared by a variety of methods as well, including those based on IL.

The use of IL allows such nanostructured separation matrices to be produced easily and very inexpensively. Nanostructures in which channels are on the order of the excluded size of dissolved biomolecules allow an enhanced flexibility in separation. Higher resolution may be obtained in combination with any of the following mechanisms namely, size exclusion, electrophoretic mobility, isoelectric point, asymmetric diffusion, entropic trapping, hydrophobic interaction and affinity interaction (molecular recognition), as well as others. The gradient matrices produced allow efficient separation and identification of biomolecules such as native proteins and protein complexes in addition to denatured proteins and nucleic acids.

Nanolithography-generated systems have advantages over conventional systems in terms of (1) the virtually perfect uniformity of pore size and pore size distribution from device to device, and (2) the flexibility to precisely define the required distribution (gradient) of pore sizes and pore chemistries. This high degree of reproducibility and versatility in nanofabrication will result in the ability to construct separation devices that exhibit unprecedented degrees of flexibility (resolution, dynamic range) and reproducibility in their separation characteristics.

The separation gradient may be formed by a variety of means including, for example, nanolithography (e.g., IL, electron beam, local probe, nanoimprint) and pattern transfer (etching, deposition, lift-off) means.

FIG. 6 shows a schematic of a nanostructured gradient (chirped) separation matrix. The separation gradient may be formed by a variety of means including nanolithography (e.g., IL, electron beam, local probe, nanoimprint) and pattern transfer (etching, deposition, lift-off) means. FIG. 6 illustrates a graded array of nanostructures. The aperture size between the nanostructures approaches molecular dimensions. The arrows signify the direction of movement of molecular species comprising the mixture to be separated and the direction of separation. The height of the nanostructures is preferably sufficiently larger (e.g., 100 nm-1 μm) than the diameter to allow for higher throughput of the separated species.

Multiple-exposure IL moire patterns provide for cyclic gradients that may be used for simultaneous manufacture of multiple structures. Gradients may also be fabricated across uniform patterns by non-uniform deposition or etching using properly designed deposition and/or etching tools and techniques such as oblique incidence of etch/deposition atomic/molecular species (shadowing). Analogous techniques may be used in generation of gradients in surface modification chemistry incorporated into the array.

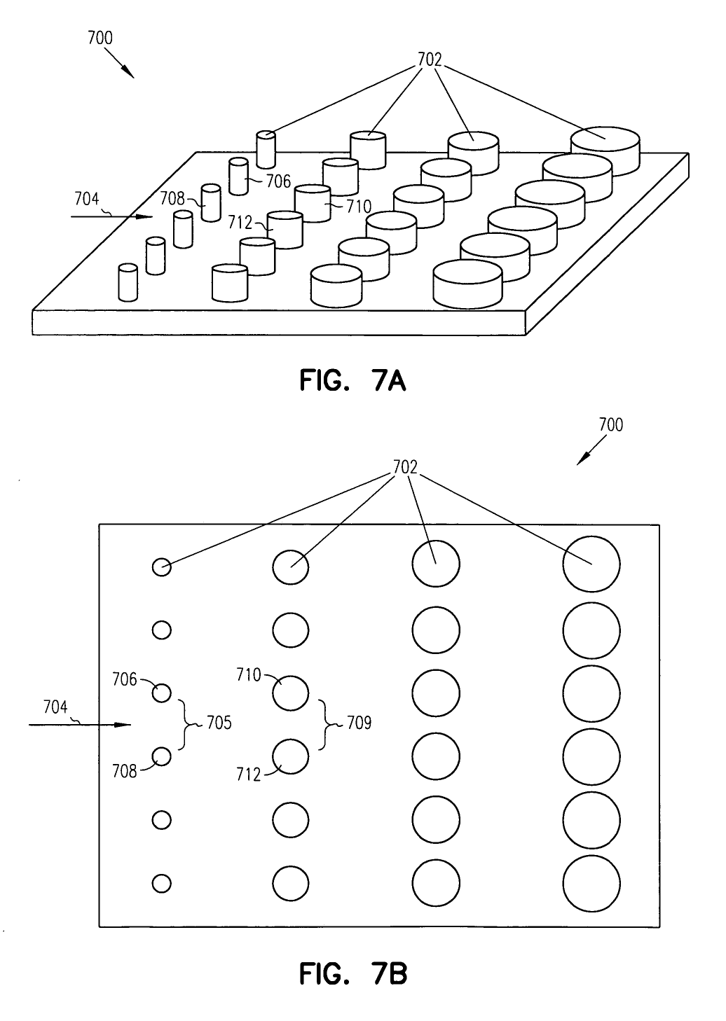

FIGS. 7A and 7B show a perspective view and a top view, respectively, of an embodiment of a nanostructured matrix. Matrix 700 has a plurality of protrusions 702. A sample containing some concentration of molecules moves in the direction of arrow 704. The diameter of channel 705 between protrusion 706 and protrusion 708 is larger than the diameter of channel 709 between protrusions 710 and 712. This change provides a gradient such that larger molecules are inhibited from moving the entire length of matrix 700 once the molecules encounter channels between two protrusions that are smaller than the diameter of the molecule. FIGS. 7A and 7B may be extended to formation of channels to delineate the pathway for molecule movement.

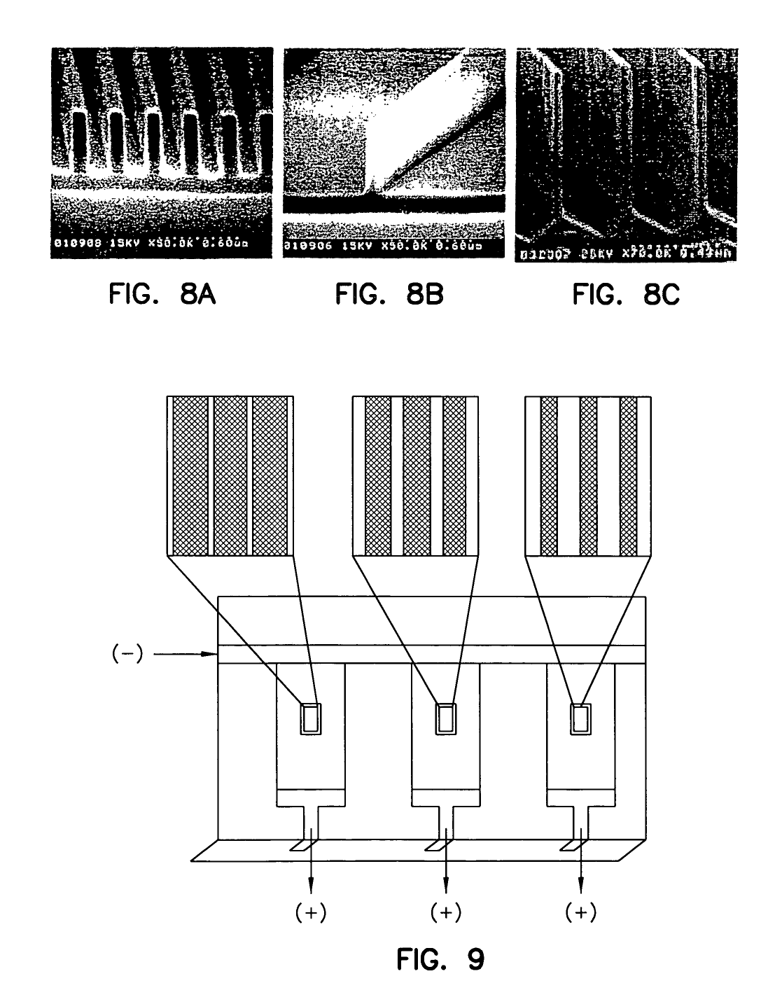

As an example of an embodiment of a channel formation, IL and anisotropic wet etching of Si allow the creation of open, parallel nanostructured channels (e.g. uncapped in the direction perpendicular to the surface) with lateral features on the order of biomolecular length scales (˜1-10 nm) but with overall dimensions reaching the microscopic (˜100 μm) or even macroscopic (˜1 cm or greater) scales. Depending upon the dimensions, molecular transport mechanisms may include diffusion, electrophoresis or bulk-flow. The relatively large vertical scale is sufficient to allow high throughput of molecules and external pumping using either electrokinetic or electro-osmotic forces. Examples of high aspect ratio IL nanostructured samples are shown in FIGS. 8A, 8B and 8C. Such architectures are applicable to channel and post arrays that are of interest for the separation of proteins and large DNA molecules.

Arrays of nanostructures (either of uniform size or with a gradient of sizes) may be surface-modified with chemical species that enhance the separation characteristics of the matrix. These chemical species may be distributed uniformly over the nanostructured separation matrix or may be distributed in a gradient (continuous or discrete) in the direction of separation over the matrix. These chemical species may include small organic molecules, polymers, receptors or other biomolecules.

IL may be used to expose patterns on photoresist on silicon or other materials (which are later etched). Silicon and some other materials may have an oxide surface that is easily modified with silanization reagents. Synthetic strategies for modification are also available for other materials (besides oxides), including native silicon and noble metals (e.g., gold). Monomolecular layers may be created from a wide range of commercially- or synthetically-available chemical species that will enhance separation characteristics based on the type and degree of interaction of chemical species being separated with the walls of the surface-modified nanostructured separation matrix. Examples of types of surface modifications (either as gradients or uniform) include the use of hydrophilic oligomeric and polymeric species e.g., poly-ethylene glycol (PEG) to minimize interactions of chemical species especially proteins, with nanostructured surfaces; use of hydrophobic molecular or oligomeric species to elicit hydrophobic interaction of chemical species (esp. proteins) with nanostructured surfaces; use of mixtures of hydrophobic and hydrophilic species (polar, apolar, H-bonding, ionic) to tune interaction of different chemical species with surfaces; use of ionic molecular species and mixtures of ionic species to tune interaction of different chemical species with surfaces; use of biomolecular or organic receptors to elicit molecular recognition of small molecules, polymers, proteins, DNA, RNA, or oligonucleotides with the surface; use of oligonucleotide probes to tune interactions of DNA, RNA or nucleic-acid binding proteins with the surface; use of cyclodextrins, macrocyclic antibiotics, crown ethers and other chiral selectors to tune enantiomeric interactions of chemical species with the surface; and use of stimuli-responsive (smart) molecules or polymers to allow external control of interaction of chemical species with the nanostructured surface.

Other embodiments of types of separation systems may be thought of as having discontinuous gradients. These separation systems contain areas with different aperture sizes, and may be made either by separate exposures at different intensity, at different pitches through shadow masks, or by using multiple exposure techniques to eliminate rows and/or columns of pillars. Such systems are especially useful in that they will allow recovery of separated compounds (purification). An example of a schematic of such a design is presented in FIG. 9. A mixture of negatively charged biomolecules (e.g., SDS treated proteins or DNA) is loaded at the left, top corner of the chip, and is driven electrophoretically across a series of discrete “sieves” that have increasing aperture size, such that smaller, and then larger molecules pass through the consecutive sieves. Each sieve is connected to a separate outlet port, such that different sized biomolecules may be collected at different outlets. If necessary, these attachments may be made through the top or bottom of the chip, and additional separation in this direction may then be combined with recovery. More sophisticated designs allow continuous purification and sample recycle.

Microfabricated Integrated Multi-Dimensional, Multi-Technique Separation Systems

Various embodiments allow a variety of different separation strategies (electrophoresis, iso-electric focusing, affinity chromatography, hydrophobic interaction chromatography, enantiomeric resolution) to be used on a single monolithic device, thus allowing for ease of use and compactness of instrumentation.

The closest existing commonly used multi-technique separation is two-dimensional gel electrophoresis (2DGE). In traditional 2DGE, proteins are first separated according to isoelectric point, followed by resolution by mass-to-charge-ratio using standard polyacrylamide electrophoresis. This process requires that two separate electrophoretic procedures be performed, each requiring manipulation of the sample. A nanostructured matrix of the present invention allows for sequential analysis on a single chip, thus reducing sample loss and diffusion. The wide range of chemical modifications and array architecture allowed by IL devices will also permit separation of proteins by means in addition to size and isoelectric point, either by appropriate chemical patterning and valving of the device, or by addition of a third separation and/or dilution dimension.

In some cases, the open nanostructured channels may be sealed in order to provide closed ducts, through which solutions may diffuse or be pumped. This may be done by bonding a “roof” to the wafer containing the open nanostructured channels to form closed channels. There are several methods available (currently in use for microscale devices) that may be explored. One alternative is a bonding procedure that uses sodium silicate (deposited through spin-coating) as an adhesive, which may be cured at room temperature overnight. This method used on glass substrates results in mechanical strengths comparable to high temperature bonding techniques.

A second alternative is to use a molecular bonding process. Silane monolayers would be formed on both the tops of the protrusions on the nanostructured channel wafer (e.g., through contact printing) and the polished “roof of the channels. The silane molecules used to form the monolayers would be terminated with complementary functional groups (e.g., amines and aldehydes) such that the two silane monolayers would chemically bond. This would result in almost a single monolayer between the two surfaces, and prevent clogging of the nanostructured channels. Since this technique requires no heat and may be done in aqueous media, delicate proteins or other molecules would not be damaged during the bonding process. Finally, a “roof” may be held in place by capillary forces alone. Such a scheme may work well where low pressures flows are involved (diffusive separations, electrophoresis or electro-osmosis), but it may not be suitable for externally pumped flows.

Fabrication of separation matrices systems from materials (e.g., Si and quartz) commonly used in the fabrication of integrated circuits is advantageous. They have unique etching and surface modification characteristics that are well established, and may be easily implemented in existing microfabrication facilities for the development of complex separation and detection systems. Other materials with advantageous characteristics may also be used.

Embodiments of a nanostructured matrix may be used for two-dimensional gel electrophoresis, and a number of other separation techniques may be combined with size exclusion and/or isoelectric focussing, In addition, the matrix has the capability of expansion beyond two dimensions.

The analytical potential of a nanostructured matrix embodiment may be enhanced by combining two or more standard types of analysis on a single platform. Among the possible combinations of separation technologies applicable to this platform are those analogous to PAGE, isoelectric focusing, hydrophobic interaction chromatography, affinity chromatography, enantiomeric resolution and capillary electrophoresis. The matrix lends itself well in carrying out equivalent molecular weight separations, with either electrical currents or flow as the driving force.

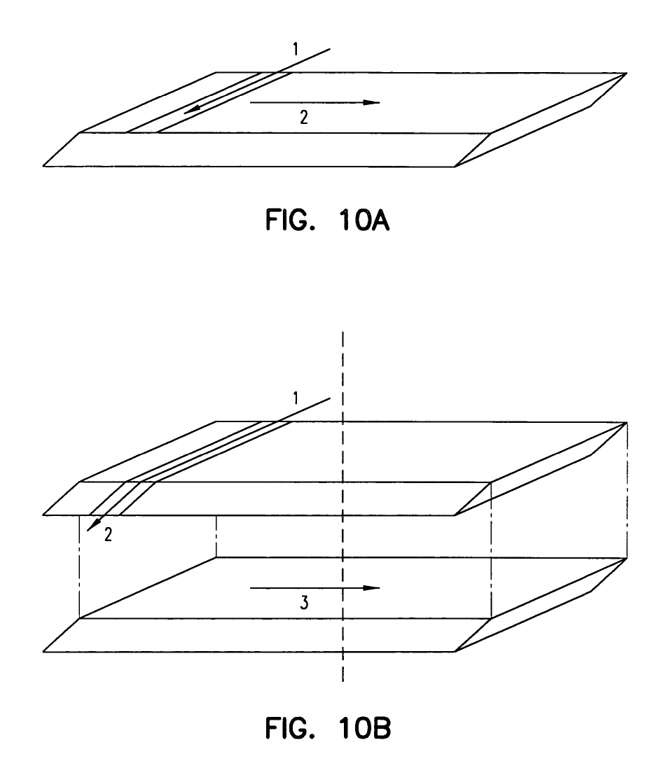

FIGS. 10A and 10B schematically depict an embodiment of a model separation system. Multi-technique separations may be performed either in the plane of a particular separation matrix (FIG. 10A) or may be performed in a multi-level structure (FIG. 10B). In FIG. 10A, molecules are separated along arrow 1 and then along arrow 2. The separation matrices corresponding to arrows 1 and 2 may be any of the types described herein. The driving force for transport along the direction of the arrows may be any of those described herein. FIG. 10B shows an exploded view of a two-technique separation in a two-level separation system. The complexity of the systems and the number of separation stages or techniques may be increased or modified as needed.

FIG. 10B exemplifies the combination of two or more gels (with or without gradients) in a multi-level, multi-stage device that allows for combinations of different separation strategies (e.g. electrophoresis, isolectric focusing (IEF), affinity chromatography, hydrophobic interaction chromatography) on a single monolithic device. For example, IEF and size exclusion may be used in a manner similar to 2DGE. These two dimensions, however, may also be combined with another dimension, for example, antibody affinity chromatography, to achieve more precise separations. The types of separations themselves may be combined in a nearly infinite variety of combinations to achieve the best possible separations for the molecules. In addition, this system allows for sequential analysis on a single chip, thus increasing efficiency of sample use.

Various embodiments are useful in proteomics by enabling combinations of different types of analysis on a single chip, e.g. size exclusion in one dimension with chemical differentiation in the second. A third dimension, oriented perpendicular to the two dimensional array on the chip, may then be used for further separation, or for recovery and further characterization of isolated spots.

Various embodiments may find use in protein separations for forensic and medical diagnostic tools and in the separation of bioengineered proteins. Forensic analysis and diagnostics, for example, depend heavily upon differentiation between carbohydrate moieties on blood proteins and bacterial cells. Discovery of clinically useful drugs often depends on identifying interactions with specific cellular receptors, which are usually glycoproteins. Capillary electrophoresis has been extremely useful in separations of acid carbohydrates, with derivatization of the column. Various embodiments allow for the separation of two properties, for example glycoprotein size and carbohydrate content on a single platform, thus eliminating the need for cumbersome recovery between steps and increasing the yield of useful analyte.

Recently, techniques utilizing antibody-based affinity separations have transitioned from clinical laboratories to those for environmental monitoring. Various embodiments allow sequential analysis of at least two different properties, thus increasing sensitivity of analysis, with particular interest for environmental monitoring.

Various embodiments allow for separation of a variety of sizes of nucleic acid species, and thus, may be used for separations that are currently done by standard and pulsed-field gel electrophoresis, as well as nucleic acid sequencing. In addition, modification of the device by nucleic acid-binding molecules (e.g. proteins, drugs) allows for isolation of relevant target sequences from previously uncharacterized genomes, or for isolation of the biocomplex formed with the nucleic acid. Because separation may be multidimensional, these devices may be attached in series with a reaction chamber (for example, a PCR thermocycler) and the resultant product directly fed into the separation platform for purification and analysis in a single device.

IL may be used to create nanostructures on a variety of substrates. IL, in combination with other standard lithographic and microfabrication methodologies, may be used to create a variety of nanostructures which may be modified in many ways to produce tools for separation of relevant biomolecules. These have advantages over contemporary molecular separation systems because they exhibit superior performance (resolution, sensitivity, dynamic range, applicability, reproducibility), may be parallel-produced at relatively low cost, and are extremely flexible in terms of chemical modifications. They have defined features that may be reproducibly made, enable flexible and complex separation, and may be used with existing bioseparation and detection strategies.

### EXAMPLE

In a non-limiting example, a design and construction of microscale electrophoresis cells may incorporate much of the characteristics of an embodiment into a compact system. The cell preferably has the following characteristics: (1) electrochemical current and fluid flow must be restricted to occur only through the separation matrix; (2) loading and stacking functions must be included; (3) monitoring of mobility and biomolecular detection must be possible (e.g., through fluorescence imaging); and (4) for certain applications, separated compounds must be recoverable. Simple methods have been used for incorporating nanostructured silicon/silica chips into electrophoresis cells that satisfy criteria (1-3) above. For example, simple methods of rapid prototyping of elastomeric gasket materials have been used. FIG. 11 presents a schematic of a simple electrophoresis cell design. The cell design allows formation of a electrophoretic nanofluidic system that incorporates a nanopatterned oxidized silicon chip of arbitrary dimension and arbitrary nanofluidic design. Thus, the feasibility of use of chips with nanostructured surface features that have been prepared using IL has been established. Using such a simple cell, the experiments have demonstrated that electrophoretic mobility may be used to transport proteins through nanostructures formed through IL lithographic patterning of silicon wafers. Protein loading was achieved through tubing attached to the electrophoresis cell. Uniform stacking of the proteins against the nanostructured chip may be achieved through optimization of the geometry of the loading tube with respect to the chip. Gas bubbles that evolve at the electrode surfaces may be restricted from entering the separation matrix by a hydrogel membrane.

Integrated Nanofluidic Chips

Embodiments for the fabrication of nanoscale structures with dimensions approaching the scale of biological molecules offers entirely new approaches to the study of fluid dynamics and biomolecular transport. Ultimately, a parallel lithographic approach will be necessary if devices based on these nanofluidics are to achieve widespread availability and acceptance. An embodiment provides a flexible, all-optical lithography alternative that is amenable to large-scale production. In an embodiment, interferometric lithography (IL) and anisotropic etching produce large areas of parallel, nanofluidic channels with widths of ˜100 nm and depths of up to about 500 nm. In an embodiment, interferometric lithography (IL) is used with a 355 nm frequency-tripled Nd-YAG light source. In an embodiment, standard optical lithography is used to create interfacing microchannels, such that the range of spatial scales on one chip varies by 10⁴ (from mm scale reservoirs to about 100 nm nanochannels). Exemplary embodiments demonstrate capillary action and electrophoretic motion of fluorescent dye solutions.

The study of molecular transport phenomena in fluidic channels of nanoscopic dimensions is a current frontier in experimental and theoretical fluid dynamics. The dearth of convenient experimental systems has thus far hindered the development of devices needed for studying nanofluidics. Nanofluidic systems have a variety of applications including molecular separations, manipulation and detection of individual biomolecules, and sensors systems. The lack of convenient and readily available experimental systems has hindered the validation of theoretical and simulation studies that have predicted unique transport properties and molecular dynamics in such systems. Fabrication techniques of such systems need to be amenable to high throughput production, allow nanoscale patterning over large surface areas, facilitate integration of nanofluidic components to the micro- and macroscale components, permit flexibility in design, and employ materials that are compatible with biomolecules. Embodiments herein describe and demonstrate a convenient and versatile method for fabricating nanofluidic systems that is based on large-area optical lithography techniques. Such embodiments are well suited for fabrication of complex fluidic systems that integrate microfluidic and nanofluidic channels for study and manipulation of solutions.

Both reductive (top-down patterning) and synthetic (e.g., self-assembly) fabrication methods are being investigated for the formation of nanofluidic systems with well defined and controllable channel/pore sizes, with the reductive approaches thus far showing the greatest promise for integration into complex fluidic systems. The most commonly used method for reductive fabrication in producing nanofluidic systems with well defined and controllable feature sizes has been based on electron-beam (e-beam) lithography, which has the capability for exquisite resolution and versatility in the formation of complex patterns. However, e-beam lithography is a serial technique that is slow and thus inherently not well suited to the formation of large numbers of nanotextured surfaces extending over macroscopic areas, such that high-throughput fabrication is not practical with e-beam lithography.

Nanoimprint lithography has been recently demonstrated as a novel alternative fabrication approach. In these approaches, a mold is formed first, usually with e-beam lithography for nanoscaled features. Optical lithographic techniques have been used to introduce larger features into the mold, such as interfacing gradient structures. The pattern is transferred to a thermoplastic polymer, typically polymethylmethacrylate (PMMA), through heat and pressure or into an ultraviolet (UV)-settable liquid or into UV-polymerizing liquid. Nanoimprint lithography approaches are parallel, fast, and suited for creating nanotextured patterns over macroscopic areas on chips with some restrictions on the range of spatial frequencies in the pattern. However, a new mold must be created whenever a feature characteristic needs to be changed (e.g., pitch, channel size, gradient scale, etc.). Additionally, nanoimprint techniques have some difficulty in accommodating wide ranges of feature size into a single pattern. The issue of stamp lifetimes has also not yet been fully explored and may pose a challenge to high-throughput fabrication.

Various embodiments for fabrication use optical lithography, which is well developed, reliable, flexible, and capable of extremely high throughput fabrication. Traditional optical lithography has been used by others for the fabrication of step-shaped nanofluidic devices on silicon where the structures are nanoscopic in one cross-sectional dimension only (the vertical direction, controlled by deposition or etching, rather than the transverse dimension, controlled by lithographic pattern formation). Various embodiments provide for making nanofluidic channels that are nanoscopic in both cross sectional dimensions and have repeatable, highly ordered structures over macroscopic surface areas with easily varied feature dimensions (such as pitch size and channel width), created with a process that is fast and relatively inexpensive.

Interferometric lithography (IL), a maskless technique based on the interference of two or more coherent beams, allows one to inexpensively and quickly pattern nanoscopic features over large surface areas with easily varied feature dimension (e.g., pitch size and channel width). It is well suited to high-throughput manufacturing. It may be combined with traditional optical lithography to yield a wide range of characteristic sizes (mm's to nm's) on a single device.

With IL, two coherent light beams of wavelength λ are crossed at an angle 2θ, producing a regular interference pattern with d=λ/(2×sin θ) describing the period. With an UV light source [such as third harmonic light from a Nd YAG laser (λ=355 nm)], one can easily obtain periods on the order of hundreds of nanometers and transverse pattern features in the sub-100 nm range, well beyond the scales available from traditional optical lithography approaches. In an embodiment, deeper ultraviolet sources and immersion techniques may extend these scales to sub-100 nm periods and ˜10 nm channel widths (all over macroscopic areas).

With this approach, nanochannels can be etched into silicon, rather than being pressed into plastic. Silicon can be easily oxidized after etching, providing a surface for the nanochannels that is inert, electrically insulating, and hydrophilic (characteristics that are very important for biofluidic applications). Silicon oxides can be chemically functionalized with silane chemistry. A variety of etching processes and wafer-bonding techniques are available for silicon. This is important because most nanofluidic devices require one to seal the tops of nanotextured surfaces to form nanoscopic tunnels rather than trenches.

Nanotexturing a surface is only one part of the nanofluidic fabrication problem. For most fluid dynamics studies it is necessary to seal the tops of the nanotextured surface to form nanoscopic tunnels rather than trenches. Others have sealed nanotextured surfaces with wafer bonding techniques such as anodic bonding with silicon substrates and HF bonding with fused silica substrates. Patterned systems in PMMA have been sealed from the top with shadow deposition of dielectrics or with thermal bonding to another plastic sheet.

Anodic bonding depends on the application of heat, which increases the mobility of alkali ions in the glass (typically Pyrex), along with the application of a strong electrical field inside a parallel plate capacitor. The electric field causes sodium ions to migrate towards the cathode and creates a depletion region at the anode, concentrating the applied field in the region of contact between the glass and the substrate. When the temperature is lowered to room temperature (with the field still applied), the ions are immobilized, resulting in a strong bond between the glass and the wafer. The electrostatic force creates a large, uniformly distributed pressure between the capacitor plates given by (V²ε)/(2d²) where V is the voltage, ε is the dielectric permittivity of the glass, and d is the charge separation distance. Since V is on the order of a kilovolt and d is less than a μm (sodium ion depletion distance in the glass), very large pressures are exerted. In an embodiment, anodic bonding is applied to a 2D, grating-like nanotextured substrate as opposed to a step-like nanofluidic device.

IL may be combined with traditional optical lithography to yield a wide range of characteristic sizes (mm to nm) on a single device. In a non-limiting example of an embodiment, the fabrication of two variations of nanofluidic devices is described herein. The first example device is a large-area nanochannel array chip having an grating with a roof. In an embodiment, the grating includes an oxidized grating. In an embodiment, the nanochannel array is configured consisting essentially of an oxidized grating with a bonded Pyrex roof. In an embodiment, the roof includes a bonded Pyrex roof, which has holes drilled in it for convenient introduction of fluids. In an embodiment, the large-area nanochannel array chip consists essentially of an oxidized nanoscale Si grating. In an embodiment, the Pyrex roof is anodically bonded. While easier to make, it was more complex to interface this chip to external, macroscopic fluidics. The second example device contains a limited area of nanoscale features, integrated microchannels, and macroscopic reservoirs, using a convenient cross configuration that interfaces the nanofluidics to the macroscopic world and provides control mechanisms for fluid flow. In an embodiment, the second device contains a limited area (1×10 mm²) of nanoscale features, integrated about 200 μm wide microchannels, and ˜2 mm wide reservoirs. Such cross configurations have been successfully and widely used in study and manipulation in microfluidics. For example, the formation of a compositional plug or band in microchannels is facilitated by the cross configuration. In an embodiment, a band is formed in a well-defined nanofluidic channel array. Others have also used microchannels as an interface but used simpler, linear configurations.

For both example chip designs, silicon <100>wafers are cleaved into small (3×4 cm²) chips. These chips may be cleaned in piranha solution (1 part H₂O₂, 2 parts H₂SO₄ by volume), triple-rinsed with deionized (DI) water, dipped in HF acid (to remove the native oxide layer and any remaining inorganic contaminants), and again triple-rinsed with DI water. About 150 nm thick layer of XHRiC-16 (Brewer Science, Inc.) anti-reflective coating (ARC) is spin-deposited and hard baked at about 175° C. for about three min. About 200 nm layer of positive photoresist [SPR510a photoresist diluted by an equal amount of EC-11 solvent (Shipley, Inc.)] is spin-deposited, and soft-baked at about 95° C. for about three min. Each layer may be spun on at about 4000 rpm for about 30 sec.

The frequency-tripled (λ=355 nm) output of a YAG-Nd laser (Infinity 40-100, Coherent Inc.) may be used as the exposure light source. The laser beam is expanded and illuminates a right-angle reflector assembly 1200 having a mirror 1205 as shown in FIG. 12. Third harmonic Nd YAG laser light (represented by the arrows) enters the corner cube reflector assembly and interferes with itself on the surface of the chip 1210. The grating pitch can be changed by rotating the assembly relative to the incoming laser light, thus changing the angle 2θ between the light rays. The chip, which may be a Si sample, is held to the reflector assembly with a vacuum chuck 1220. Although the example focuses on pitches of about 500 nm (θ˜26°), the reflector assembly 1200 can be rotated to produce a variety of grating pitches.

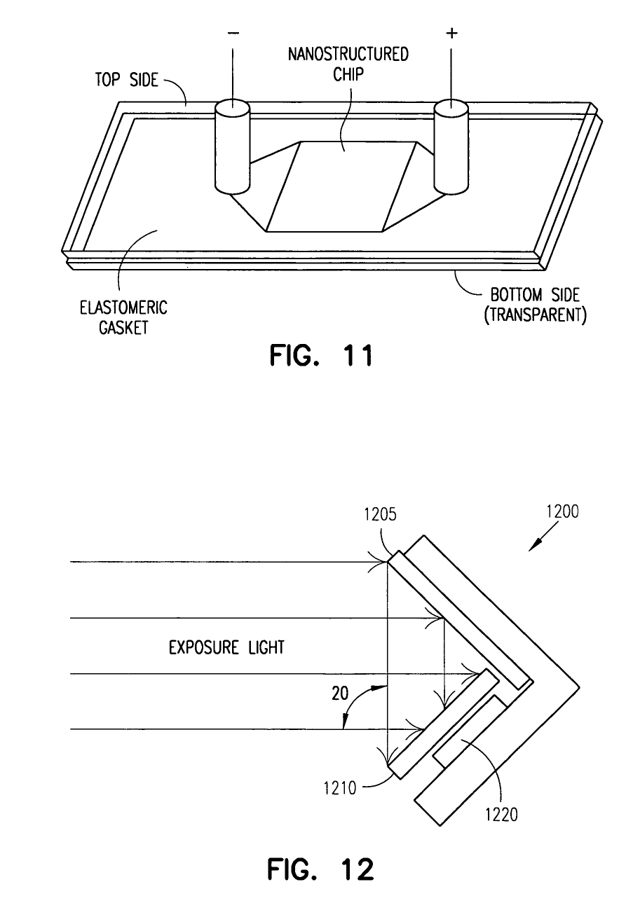

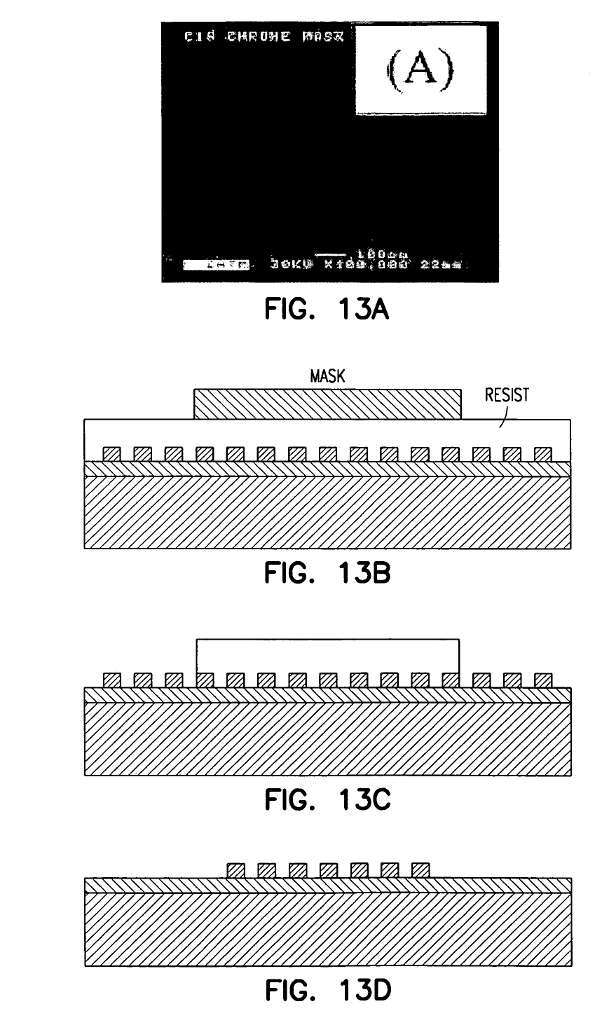

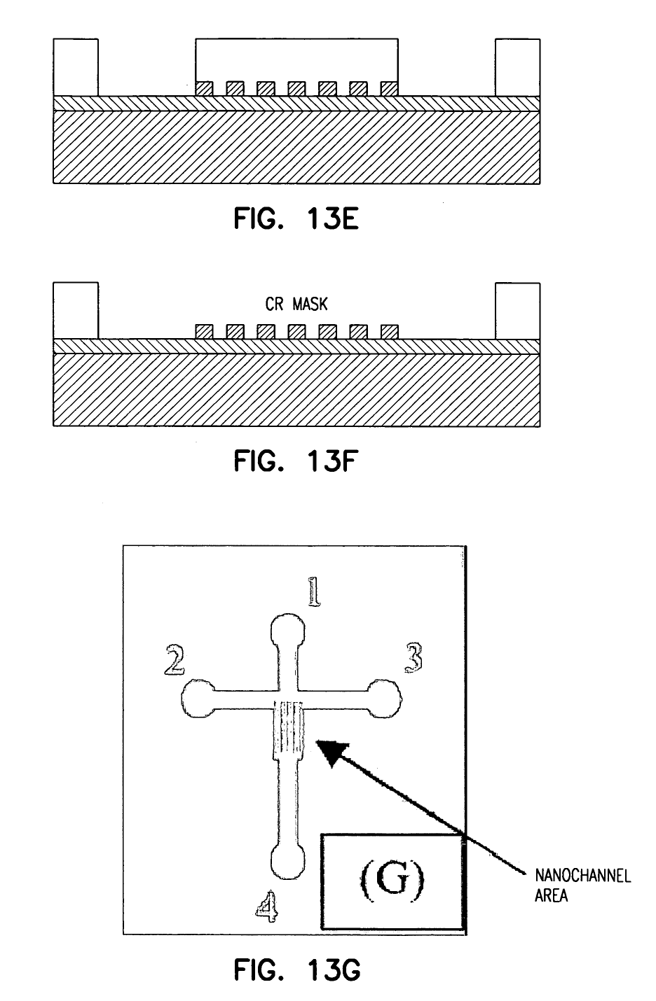

After exposure, each sample chip may be soft baked at about 110° C. for about 1 min, developed using undiluted MF702 developer (Shipley, Inc.), and rinsed with water, leaving a photoresist grating. The developed chip is placed in an e-beam evaporator where a thin (about 35-40 nm) layer of Cr is deposited. The remaining photoresist (and the Cr on top of it) may be lifted off using an airbrush acetone spray, leaving a negative-tone Cr etch mask layer on top of the remaining ARC (which is impervious to the acetone which does not dissolve in acetone). A field-emission scanning-electron microscope (FE-SEM) image of one such mask is shown in FIG. 13A. For the large-area nanochannel array device, these chips were ready for reactive-ion etching (RIE). The more complex design, with the integrated microfluidics, required additional processing steps before etching.

Additional processing steps may be used to prepare the integrated microfluidics on the more complex samples before etching. To form the 200 μm wide microchannel interfaces and the 2 mm diameter reservoirs, about a 1.44 μm thick layer of AZ 5214-E (Shipley, Inc.) resist may be first spun at about 3000 rpm for about 30 s onto the samples with Cr etch masks. The chips may be then exposed with a rectangular exposure mask, using a conventional proximity aligner, developed, and baked (about 95° C. for about 10 min) to produce a protective layer over the area where the nanochannels are intended to be retained, as shown in FIG. 13B. The chips may be placed in CEP-200 Cr etchant (Microchrome Technology) to remove the unprotected regions of the Cr mask, then the protective photoresist layer may be removed with acetone leaving a small area (about 1×10 mm²) on the chip with a Cr etch mask [FIG. 13C]. About a 1.44 μm thick layer of AZ 5214-IR (Shipley, Inc.) resist may be spun over the chips, then exposed with both the microchannel exposure mask [FIG. 13E] and a second rectangular exposure mask to remove the resist from the nanochannel area [FIG. 13D]. This results in a chip with an etch mask made of photoresist and a Cr grating as represented in FIG. 13F, which may provide a photoresist etch mask for the microscale features and a Cr etch mask for the nanoscale features, as represented in FIG. 13G. FIGS. 13A-13G show mask fabrication including: (A) FE-SEM image of a Cr etch mask after lift-off; (B) photoresist is spun over the Cr grating and exposed using an intensity mask; (C) after developer the Cr mask is protected by a layer of resist; (D) after Cr etch and acetone rinse a smaller area of Cr grating remains; (E) a second resist coat is spun, exposed with microchannel mask, and developed, (F) the chip is ready for etching after an additional exposure and develop process to clear photoresist away from the Cr etch mask; (G) a top view of the final etch mask for an integrated chip with reservoir areas numbered for reference.

The samples may be reactive ion etched (RIE) using a mixture of O₂ and CHF₃. The etched silicon gratings may be cleaned with piranha solution to remove the ARC, Cr, and residual polymer from the RIE process [FIG. 14A]. After cleaning, the chips may be placed in a quartz tube furnace containing ultrahigh-purity grade O₂ at about 1100° C. for about 45-60 min to form an insulating oxide layer [FIG. 14B]. In an embodiment, the channel walls may be provided with an insulating oxide layer, rather than a silicon layer. FIGS. 14A-14C shows FE-SEM images of etched samples: (A) silicon wafer after etching, (B) the same wafer after oxidation, and (C) an oxidized grating with a bonded Pyrex roof. All samples may be sputtered with gold prior to imaging to reduce charging efforts. The scale bars indicate distances of about 100 nm.

After etching and oxidation, all chips may be capped with about 1 mm thick Pyrex No. 7740 roofs using anodic bonding. These glass plates used may have a surface quality of about 1.8 λ [measured across about a 2.54 cm diameter circle with a laser interferometer (Zygo, Inc.) at 633 mm (using a red He—Ne laser)]. The roofs for the integrated chips may be predrilled with four holes (˜2 mm diameter), located above the circular reservoirs at the ends of the microchannels. These four holes may be formed with one on each edge of the roof. These holes may provide access for standard pipettes. The roofs for the large-area nanochannel chips may be provided with four holes (about 3 mm diameter) drilled in a row on one edge to provide ports for convenient loading of fluorophores and/or other solutions.

The bottom electrode of the bonding apparatus may be configured as the grounded metal surface of a hot plate that supports the oxidized silicon sample. The Pyrex roof is placed on top of the oxidized silicon chip. The upper electrode, as a small aluminum block, may be placed on top of the Pyrex roof and connected to a high voltage dc power supply. An example power supply is a 205A-03R power supply, Bertan associates, Inc. The temperature of the hot plate may be raised to about 380° C. before charging the capacitor structure. In an embodiment, the temperature of the hot plate may be raised to about 380° C. before charging the capacitor structure by increasing the voltage in steps while monitoring the current. The capacitor voltage typically may be increased slowly until the current reaches a value of about 1-2 mA, allowing the current to decay to ˜0.5 mA (indicating the formation of a space charge blocking layer) before further increasing the voltage. A voltage as large as possible may be used while avoiding arcing between the electrodes (typically about 800-1000 V). The current flow through the system may be monitored until it decays to ˜100 μA per chip before shutting off the heating element and allowing the sample to cool. The voltage may be decreased to zero when the temperature drops below about 150° C. A FE-SEM image of a bonded chip cross section is shown in FIG. 14C.

The liquid storage volume of the holes in the glass roofs is rather small, leading to problems with evaporation and difficulty in loading. To compensate for this, small (volume −50 μl) plastic reservoirs (made by cutting pipette tips) may be attached to the glass with ordinary epoxy.

In non-limiting examples of operating the example embodiments, a solution of standard Tris/glycine electrophoresis buffer (about 0.24 mM Tris and about 1.92 mM glycine, pH 8.8) may be prepared. In an embodiment, this solution may then be diluted about 100× with DI water. This buffer may be filtered through about a 0.2 μm filter to remove particulate contaminants and then degassed under vacuum to reduce outgassing during electrokinetic motion. In an embodiment, the degassing may be conducted for at least about 3 hours. About a 5 mg/ml suspension of Alexa Fluor 532 C₅ maleimide (Molecular Probes) may be prepared in about a 1.5 mM Tris HCl buffer (pH 8.8).

### Fluid Flow in the Large-Area Nanochannel Array Chip Example Embodiment

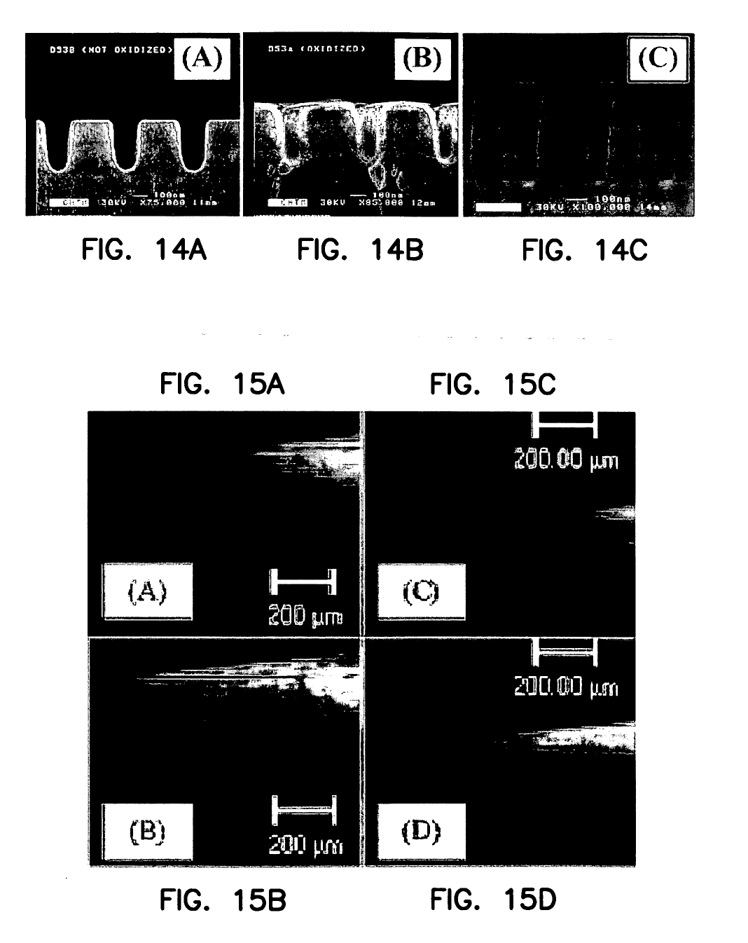

The example large-area nanochannel array design may be mounted into a Teflon chuck with reservoirs at each end of the chip. Platinum wire-mesh electrodes may be inserted into the sides of both reservoirs (˜3 cm from the chip edge). Poly(dimethyl-siloxane) may be used to secure the chip into the chuck and seal the system both for fluid flow and for the electrical isolation. The assembly may be imaged using an upright, laser scanning (543 nm) confocal microscope (Axioskop using an LSM5 scanning head, Zeiss, Inc.). The fluorescence output may be passed through a long-wavelength pass optical filter (about 560 nm cut-off). Approximately 2 μl of Tris/glycine solution may be added to each hole. After capillary action causes the buffer to move ˜5 mm through the nanochannels, a few μl of Alexa 532 solution may be added to one of the holes in the glass roof and the flow of liquid due to capillary action may be imaged. FIGS. 15A and 15B show the progression of the dye solution over 30 s. The average fluid velocity (average of several measured velocities at different points on the liquid front) of this example operation is 12.2±0.6 μm/s.

After the entire chip fills via capillary action with Tris/glycine buffer, the reservoirs may be filled with buffer. About 1 μl of Alexa 532 dye may be placed into one of the four holes on the top of the chip and the electrodes biased with about 50 V. FIGS. 15C and 15D show the progression (due to electrophoresis) of the negatively charged Alexa 532 dye towards the positive electrode over a period of about 150 s. The average electrophoretic velocity (average of several measured velocities at different points on the liquid front) of this example operation is 0.77±0.03 μm/s. FIGS. 15A-15D show confocal images of fluid motion in the large-area grating design, where the scale bars indicate distances of about 200 μm, the reservoir to the left of the picture is biased at about +50V, and the reservoir to the right of the picture is grounded. (A) The bright area is Alexa 532 while the dark area corresponds to air-filled nanochannels. The ring like interference patterns are due to some surface damage in the glass roof from the bonding process. (B) about 30 sec later, capillary action has caused the Alexa solution to flow down the channels. (C) At a different location on the chip after the chip has been filled with buffer by capillary action: The bright area is Alexa 532 while the dark area is buffer in the nanochannels. (D) about 150 seconds later, the negatively charged Alexa dye has clearly moved towards the positive electrode, indicating motion dominated by electrophoresis.

### Example Embodiment of Electrokinetic Flow Through Integrated Chips

An example integrated chip may be loaded with liquid via capillary action. First, about 50 μl of DI water may be loaded into reservoir 4 (as numbered in FIG. 13G). The flow of water through the microchannel and nanochannel areas may be monitored by eye until the water had reaches the end of the nanochannels. At that point, about 50 μl of DI water may be introduced into reservoir 3, filling the remaining three microchannels. After allowing the system to equilibrate for about 30 min, about 50 μl of Tris/glycine buffer may be added to reservoirs 1 and 2 and reservoirs 3 and 4 may be topped off with buffer. Platinum wire electrodes may be inserted into all four reservoirs and the assembly imaged with a confocal microscope. The reservoir 2 electrode may be grounded. Three independent power supplies (common ground) may be connected to the remaining three electrodes.

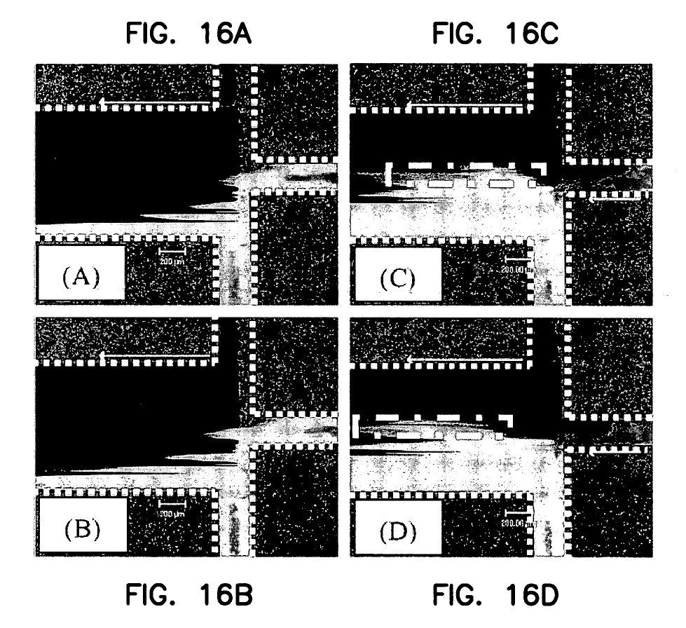

Alexa 532 dye may be introduced into reservoir 3 and its electrode biased at about −100 V. The remaining electrodes may be at ground potential. The negatively charged dye moves towards the center of the chip, entering both the nanochannel area and the microchannel connected to reservoir 1, as seen in FIG. 16A. FIG. 16B shows the juncture about 60 s after FIG. 16A was recorded. Since the dye enters the nanochannels closest to reservoir 3 first, it has progresses the farthest in those (the lowest) channels.

The bias for reservoirs 1 and 3 to about −100 V may be changed while reservoir 2 remained at about 0 V. This flushes the dye out of microchannel 1 and pinches off a slug of dye in the nanochannels across from microchannel 1, as seen in FIG. 16C. FIG. 16D shows the clear progression of the slug down the nanochannel area after about 20 s. The velocity of this slug is calculated to be 26.5±0.9 μm/s. FIGS. 16A-16D show confocal microscopy of electrophoresis in integrated chip. The channels are outlined with dotted lines for clarity, the left hand side of each picture contains the nanochannel area, and microchannels are on the upper, lower, and right hand sides of each picture. The scale bars indicate about 200 μm distances. (A) The electrode for the lower channel is biased at about −100V while the other three remain grounded. Electrophoresis causes the dye to flow through the lower microchannel towards the top part of the picture. It begins to flow down the nanochannels and the microchannel on the right as it reaches them. (B) about 60 sec later dye has progressed down the nanochannels. (C) This picture was taken after the electrode for the channel on the right hand side of the picture is biased at about −100V (along with the lower channel). The dye has been flushed out of the right hand side microchannel and pinched off a slug of dye (outlined with broken lines) in the nanochannels directly opposite the right hand side microchannel. (D) about 20 sec later the slug has progressed down the nanochannels area.

These example applications of embodiments demonstrate that the designs of these embodiments are suitable for experiments studying electrokinetic motion in nanoscale channels. It is interesting to note that much higher electrophoretic velocities in the example integrated chip were able to be achieved. This is due, in part, to the difference in nanochannel lengths: The length of the nanochannel area of the example integrated chip is about ⅓ the length of the sealed nanochannels in the example large-area chip. The microchannels are less than about ⅔ of the length of the sealed channels, and have a lower resistance (due to the larger cross-sectional area), resulting in a larger electric field across the nanochannels for a given electrode bias voltage. This, coupled with a twofold higher electrode bias, may account for about a factor of 6 difference, less than the factor of 32 observed. Another contributing factor may be the difference in buffer concentrations between the two solutions. It is also interesting to note that the fluid velocities due to capillary action in the large-area chip example are also quite fast (approximately half that of the electrbphoretic velocities in the integrated chips). Further investigation may be used to fully characterize the fluid-flow characteristics in these nanochannel samples.

Application of about 100 V to the large-area nanochannel array chips quickly results in a drop of current across the chip and an eventual cessation of electrokinetic motion, which is caused by outgassing of the solution. Indeed, rapid generation of microscopic bubbles may be seen at the edge of the glass on these chips. These effects may not be seen in the integrated chip examples, possibly because of the much better defined macro-micro-nano interface hierarchy.

In various embodiments, functional nanofluidic chips based on all-optical lithographic processes with feature sizes ranging from <100 nm to about 2 mm on a single chip may be created. IL is the basis of the nanochannel fabrication, and allows for flexible nanopatterning of silicon chips over large surface areas. Traditional optical lithography provides convenient microfluidic structures for interfacing with the nanochannels and controlling fluid flow as demonstrated with the example cross-type microfluidics. Anodic bonding allows for the sealing of a glass roof to the oxidized, patterned silicon chip to create finished devices compatible with electrokinetic motion. In various embodiments, such techniques are suited to high-throughput manufacturing, provide flexible nanotexturing over large areas, and may produce a broad range of feature sizes on a single chip. They allow one to use inert and hydrophilic nanotextured surfaces (oxidized silicon with glass roofs) that are compatible with electrokinetic studies.

Various embodiments include the addition of integrated microelectrodes to the chips, smaller nanochannel pitches, two-dimensional patterning of the nanochannels (e.g., introducing gradients in channel widths), and adding additional switching capabilities to form more complex micro/nanofluidic arrangements. In an embodiment, IL may be used to provide smaller nanochannel pitches with sub-100 nm period gratings. Various embodiments may be applied to detailed parametric studies of electrokinetic motion and flow rates of biomolecular species and to investigations of separations of biomolecular species within the nanochannel devices. Various embodiments may be used with proteins in these chips.

Although specific embodiments have been illustrated and described herein, it will be appreciated by those of ordinary skill in the art that any arrangement that is calculated to achieve the same purpose may be substituted for the specific embodiments shown. This application is intended to cover any adaptations or variations of embodiments of the present invention. It is to be understood that the above description is intended to be illustrative, and not restrictive, and that the phraseology or terminology employed herein is for the purpose of description and not of limitation. Combinations of the above embodiments and other embodiments will be apparent to those of skill in the art upon studying the above description. The scope of the present invention includes any other applications in which embodiment of the above structures and fabrication methods are used. The scope of the embodiments of the present invention should be determined with reference to the appended claims, along with the full scope of equivalents to which such claims are entitled.

What is claimed is:

1. A method comprising:
producing a regular interference pattern in a substrate using two coherent light beams to form a nanofluidic device having a pattern with nanoscopic features in at least two dimensions.

2. The method of claim 1, wherein to form a nanofluidic device having a pattern with nanoscopic features includes forming the nanofluidic device with nanoscopic vertical dimensions and transverse pattern features of less than 100 nm.

3. The method of claim 1, wherein to form a nanofluidic device having a pattern with nanoscopic features includes forming the nanofluidic device with vertical dimensions less than 10 nm.

4. The method of claim 1, wherein to form a nanofluidic device having a pattern with nanoscopic features includes forming the pattern with varied feature dimensions over a surface area of the substrate.

5. The method of claim 1, wherein producing a regular interference pattern in a substrate using two coherent light beams includes using an ultraviolet source to produce a coherent light beam.

6. The method of claim 1, wherein the method includes:
integrating microchannels in the substrate; and
forming a cross configuration to interface the microchannels to the nanofluidic device.

7. The method of claim 1, wherein to form a nanofluidic device having a pattern with nanoscopic features includes forming a Si grating with nanoscopic features in the substrate.

8. The method of claim 7, wherein the method further includes:
oxidizing the Si grating; and
forming a roof to the Si grating.

9. The method of claim 8, wherein forming a roof includes anodically bonding a Pyrex roof to the Si grating, the Pyrex roof having holes to introduce a fluid into the Si grating.

10. The method of claim 8, wherein the method further includes chemically functionalizing a surface of the oxidized Si grating with silane chemistry.

11. An apparatus comprising:
a substrate; and
a nanofluidic device in the substrate, the nanofluidic device having a structure that is nanoscopic in two dimensions.

12. The apparatus of claim 11, wherein the substrate is a Si substrate.

13. The apparatus of claim 11, wherein the nanofluidic device includes nanochannels having inert surfaces.

14. The apparatus of claim 11, wherein the nanofluidic device includes nanochannels having electrically insulating surfaces.

15. The apparatus of claim 11, wherein the nanofluidic device includes nanochannels having hydrophilic surfaces.

16. The apparatus of claim 11, wherein the nanofluidic device includes nanochannels having oxidized Si surfaces.

17. The apparatus of claim 11, wherein the nanofluidic device includes:
a nanoscale grating; and
a roof bonded to the nanoscale grating.

18. The apparatus of claim 11, wherein the nanoscale grating includes a Si grating and the roof includes a bonded Pyrex roof having holes.

19. The apparatus of claim 11, wherein the nanofludic devices includes:
microchannels; and
a cross configuration that interfaces the microchannels to the structure that is nanoscopic in two dimensions, the cross configuration adapted to provide a control mechanism for fluid flow.

20. The apparatus of claim 19, wherein the cross configuration couples to a reservoir.

21. A system comprising:
a fluid source;
a substrate; and
a nanofluidic device in the substrate, the nanofluidic device having a structure that is nanoscopic in two dimensions; and
a means to introduce fluid from the fluid source into the nanofluidic device.

22. The system of claim 21, wherein the means to introduce the fluid includes an electrode.

23. The system of claim 21, wherein the nanofluidic device includes:
a nanoscale grating; and
a roof bonded to the nanoscale grating.

24. The system of claim 23, wherein the nanoscale grating includes a Si grating and the roof includes a bonded Pyrex roof having holes.

25. The system of claim 21, wherein the nanofluidic devices includes:
microchannels; and
a cross configuration that interfaces the microchannels to the structure that is nanoscopic in two dimensions, the cross configuration adapted to provide a control mechanism for fluid flow.

26. The system of claim 25, wherein the structure that is nanoscopic in two dimensions includes Si nanochannels.

27. The system of claim 25, wherein the cross configuration couples to the fluid source.
